(19) United States
(12) Patent Application Publication
(10) Pub. No.: US 2026/0053506 A1
(43) Pub. Date: Feb. 26, 2026

(54) Secure Tourniquet Protector

(71) Applicant: Barry T. BICKLEY, Andover, MA (US)

(72) Inventor: Barry T. BICKLEY, Andover, MA (US)

(21) Appl. No.: 19/310,315

(22) Filed: Aug. 26, 2025

Related U.S. Application Data
(60) Provisional application No. 63/686,976, filed on Aug. 26, 2024.
(60) Provisional application No. 63/686,977, filed on Aug. 26, 2024.
(60) Provisional application No. 63/709,651, filed on Oct. 21, 2024.

(51) Int. Cl. A61B 17/132; A41D 13/12

(52) U.S. Cl. CPC A61B 17/1322; A41D 13/1245; A41D 13/1254

(57) ABSTRACT

Described herein is a secure tourniquet protector (STP) which secures one or more tourniquets as well as other medical devices to varied body shapes and contours or a patient undergoing a medical procedure. In embodiments, the secure tourniquet protector is provided as part of a garment (e.g., either coupled to a garment or provided as an integral part of a garment). In embodiments, a patient may don a garment comprising a secure tourniquet protector at the same time they remove their clothing and are placed in hospital garb following check-in for a medical procedure.

### CROSS-REFERENCE TO RELATED APPLICATIONS

This application claims the benefit under 35 U.S.C. § 119(e) of U.S. provisional application Nos. 63/686,976 and 63/686,977 both filed Aug. 26, 2024 and U.S. provisional application No. 63/709,651 filed Oct. 21, 2024 the contents of which are all incorporated herein by reference in their entireties.

### STATEMENT REGARDING FEDERALLY SPONSORED RESEARCH

Not applicable.

### BACKGROUND

It is common to apply a pneumatic tourniquet to an extremity when performing surgery on an extremity. This tourniquet is most commonly placed at the proximal most aspect of the limb. Usually a layer of padding is placed on the skin initially. This can be layers of cotton padding such as is used beneath casts or it is often a layer of elastic stockinette. The tourniquet is applied over this padding and secured only by snuggly applying and tightening the tourniquet around the extremity. The tourniquet is not restricting blood flow at this point but it is applied in a relatively tight fashion. When it is to be used to restrict blood flow to the extremity, the pneumatic tourniquet is inflated with an automated air pump that fills the air bladder inside the tourniquet to a pre-determined pressure set by the surgeon. Once it is desired to allow blood flow again to the extremity, usually at the conclusion of the procedure, the pump is shut off and the tourniquet is deflated, releasing the pressure and restoring blood flow.

### SUMMARY

The tourniquet is wrapped around the extremity in a circumferential fashion. Many limbs are also generally cylindrical in nature and the padding and tourniquet fit well without issue. However, this is not always the case. Some extremities, at the upper arm and at the thigh, are more cone shaped and taper sharply to a steadily smaller circumference. This can make it difficult to apply and maintain in place both the padding and the pneumatic tourniquet itself. Similarly, in an obese patient having a significant amount of adipose tissue, placement and maintenance of the padding and tourniquet are often difficult. These situations are becoming more and more common with the obesity rate in the US at 40% of the population and obesity contributing to extremity issues such as osteoarthritis. It is also common that patients are not wearing underwear during surgery. This is due to several factors. Because standard underwear is not designed to be compatible with operating room conditions, the underwear itself can get caught beneath the tourniquet and cause an area of excessive pressure resulting in injury to the skin. Hygiene issues in regard to an individual patient's underwear can also present problems with cleanliness and sterility in the operating room. Without underwear, the genitals are exposed so special care must be taken to insure that they do not get caught under the edge of the tourniquet causing injury. In order to do so, it often takes 2 people to apply the tourniquet. One member of the OR staff will hold the genitals and/or any excessive adipose tissue out of the way while a second OR team member actually places the padding and tourniquet. This occupies OR personnel when they could be accomplishing other necessary tasks prior to the surgical procedure and slows the process of beginning the actual procedure.

Described herein is a secure tourniquet protector (STP) which addresses the aforementioned and other issues with application and securing of a tourniquet to varied body shapes and contours. The secure tourniquet protector described herein also helps reduce (and ideally totally avoid) injury to patients. Furthermore, the secure tourniquet protector described herein also frees up operating room (OR) staff to perform other tasks before, during and after a patient enters an OR. In embodiments, the secure tourniquet protector is provided as part of a garment (e.g., either coupled to a garment or provided as an integral part of a garment). In embodiments, a patient may don a garment comprising a secure tourniquet protector at the same time they remove their clothing and are placed in a hospital gown following check-in. That is a patient may don a garment comprising a secure tourniquet protector without assistance from any other hospital staff (i.e., without assistance from either medical or non-medical hospital personnel).

With the patient putting on the STP themselves upon check-in, there is no additional time required by hospital personnel. In embodiments, the STP garment may be worn beneath a hospital gown. In embodiments, the STP garment may be made from a knitted fabric or stitch pattern. In embodiments, the STP garment may be made from a stitch pattern. In embodiments, the STP garment may be made from a soft stretchy fabric. In embodiments, the STP garment may be made from a soft, stretchy, machine-knitted fabric. In embodiments, the STP garment may comprise cotton. In embodiments, the STP garment may comprise a stockinette material. In embodiments, the STP garment may comprise a combination of a stockinette material and other materials. In embodiments, the STP garment may comprise stockinette material (or a stockinette-type material) where the tourniquet will be applied. It is noted that stockinette material is already commonly used for padding and it is familiar to OR personnel, other materials (or a garment comprising a combination of stockinette and other materials) can also be used.

Because the STP garment is in place when a patient enters an operating room, there is no need to apply additional padding prior to placing the tourniquet so that step is eliminated from an OR procedure (e.g., eliminated from a pre-medical procedure OR procedure).

When the STP is composed entirely of a stretchy fabric such as a stockinette material, it is substantially form-fitting. In embodiments, the form fit delineates and protects the genitals. It also gathers adipose tissue within the garment making it easy to be held or otherwise constrained out of the way of the tourniquet by the same person applying the tourniquet, thus freeing OR staff from this task.

Because the STP functions as the padding for the tourniquet, the tourniquet may simply be placed in the standard accepted fashion over the STP.

### BRIEF DESCRIPTION OF DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIG. 1A is a front view of a garment having a secure tourniquet protector worn on an upper portion of a patient;

FIGS. 1B and 1C are side isometric views of the secure tourniquet protector of FIG. 1A;

FIG. 2A is a front view of a garment having a secure tourniquet protector worn on a lower portion of a patient;

FIGS. 2B and 2C are side isometric views of the secure tourniquet protector of FIG. 2A;

FIG. 3 is a front view of a garment having a secure tourniquet protector and a region of padding worn on a lower portion of a patient;

FIG. 4 is an alternate embodiment of a garment having a secure tourniquet protector worn on a lower portion of a patient;

FIGS. 5-8 are front views of a series of alternate embodiments of a garment having a secure tourniquet protector worn on a lower portion of a patient;

FIG. 9A is a front view of a peri-operative garment;

FIG. 9B is a back view of a peri-operative garment;

FIG. 10A is a front view of a peri-operative garment; and

FIG. 10B is a back view of a peri-operative garment;

### DETAILED DESCRIPTION

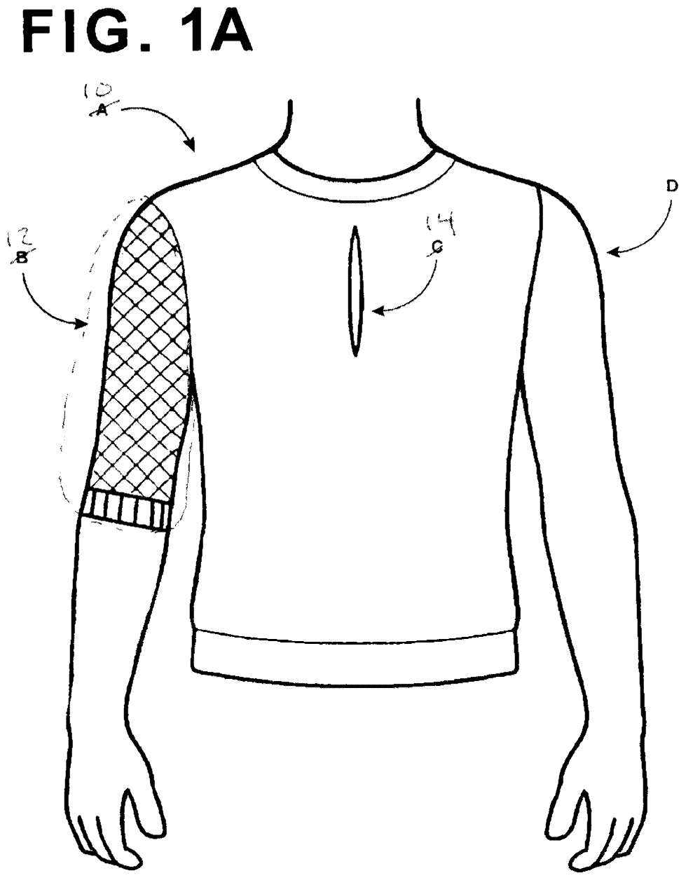

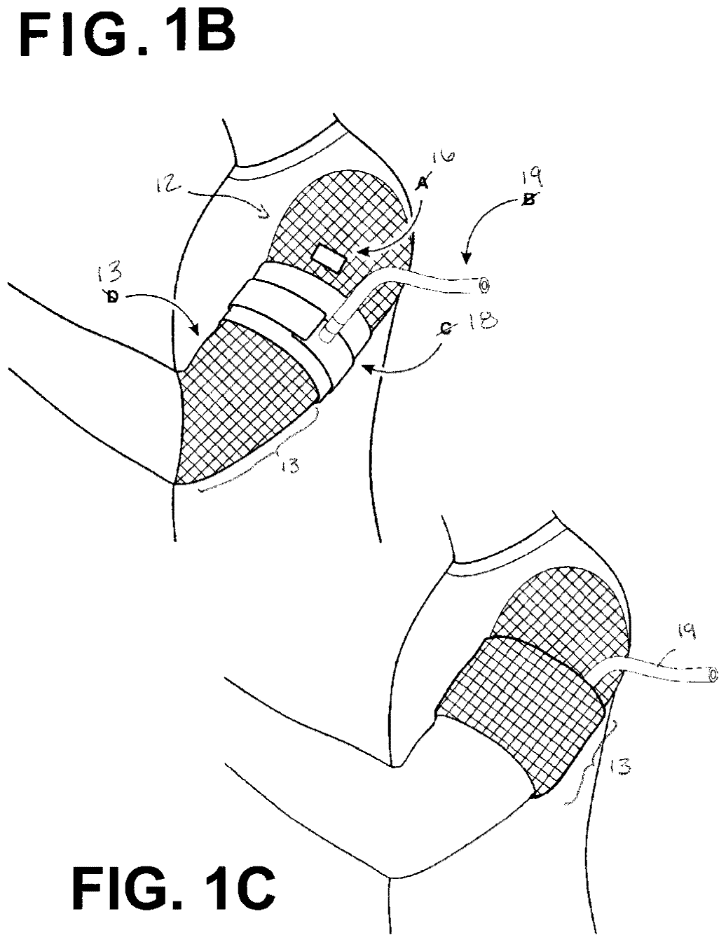

Referring now to FIGS. 1-1C in which like elements are provided having like reference designations throughout the several views, a garment 10, here a shirt-type garment, comprises a secure tourniquet protector 12 (STP). In this example embodiment, STP 12 is illustrated as a sleeve portion of the garment 10 through which a patient's arm extends. Thus, in this example embodiment, the garment is worn by a patient with the secure tourniquet protector arranged to cover an upper extremity—i.e. an arm—of the patient.

In embodiments, the STP is provided from any material having an elastic property. For example the STP may be provided from stockinette or any other knitted material or knitted fabric suitable for cleaning and/or wrapping, and/or bandaging or use in a medical environment. Preferably the STP comprises a material that provides stretch, comfort, and flexibility. Such materials include but are not limited to stockinette, spandex, latex, or rubber fibers.

In the example embodiment of FIGS. 1A-1C, the secure tourniquet protector extends from an upper portion of the patient's arm to approximately the elbow. In embodiments, the secure tourniquet protector extends from a portion of the patient at, above or below the patient's shoulder to a portion of the patient at, above or below elbow. The particular length of secure tourniquet protector 12 is selected depending upon the needs of the particular application. The secure tourniquet protector 12 is provided having a length, shape and dimensions selected to suit the needs of a particular patient and a particular application. For example, in embodiments the garment may be provided in a range of different sizes (including, but not limited to, extra-small, small, medium, large, extra-large) with appropriately sized secure tourniquet protectors. Alternatively, in embodiments, the garment may be provided in a single size with secure tourniquet protectors provided in a range of different sizes (including, but not limited to, extra-small, small, medium, large, extra-large) keeping in mind that the STP should preferably be substantially form-fitting on the patient. After reading the disclosure provided herein, those of ordinary skill in the art will appreciate how to select garment and STP sizes (e.g., an STP length, diameter, and other dimensions) to suit the needs of a particular patient and a particular application.

As used herein the term “form fitting” is intended to mean fitting close to the body part, so the shape of the body part can be clearly seen, but not tight so as to restrict a patient's circulation.

As illustrated in FIGS. 1A-1C, once a tourniquet 18 (e.g. a pneumatic tourniquet) is placed or otherwise disposed on a patient (e.g., on one or more body parts of a patient), an end of the STP (e.g., here a distal end 13 of STP 12) may be everted and folded back on itself proximally covering substantially at least the distal end of the tourniquet (and ideally, covering the entire tourniquet). Thus, in embodiments, the garment (e.g. an STP portion of the garment) extends down an extremity of a patient by a distance which allows an STP (e.g. an STP portion of a garment) to fold back over distal edge of the tourniquet and ideally over the entire tourniquet.

For additional security, once the garment is folded back on itself over the tourniquet (e.g. as illustrated in FIGS. 1B, 1C), it can be further secured by a securing means 16. The securing means may be provided as a hook and loop type fastener (e.g. a tab of Velcro® or other hook type fastener), an adhesive strip fastener or a mechanical fastener (e.g. a snap fastener such as may be provide from a pair of interlocking discs) or a button (e.g., a small disk or knob sewn or otherwise attached to a garment, either to fasten the STP being pushed through an opening (e.g. a hole or slit) made for the purpose of coupling one or more portions of the STP or other garment portion. The tourniquet is then secure and standard prepping and draping of the patient's extremity continues in preparation of beginning a surgical or other medical procedure.

The ability to fold back the form fitting elastic material portion of the garment (e.g., a stockinette portion of the garment) over the tourniquet, secures or helps secure the tourniquet from moving on the extremity (e.g., the limb). For example, the ability to fold back the form fitting elastic material portion of the garment over the tourniquet, impedes the ability of the tourniquet (and ideally, totally prevents the tourniquet) from sliding down the limb. This may be particularly advantageous in the case of an obese patient or a patient having a cone shaped extremity. Because at least the STP portion of the garment is made of a form fitting, elastic material it conforms to the tourniquet and upper end of the extremity and cannot slip distally.

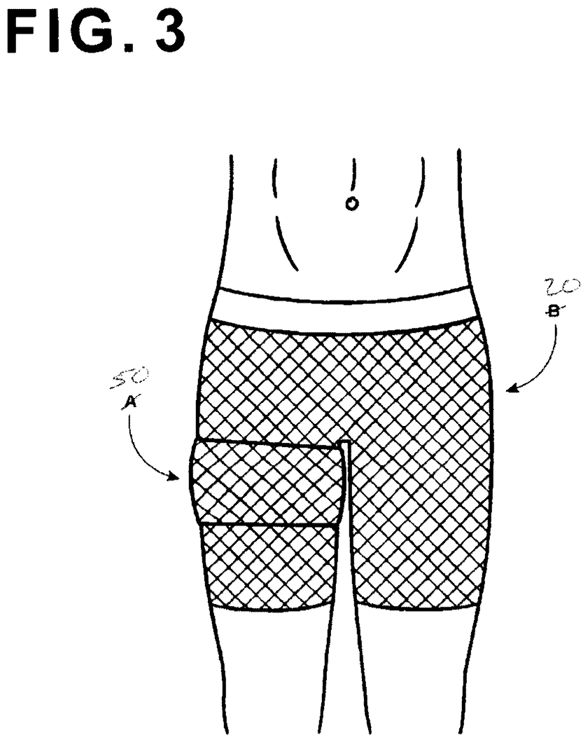

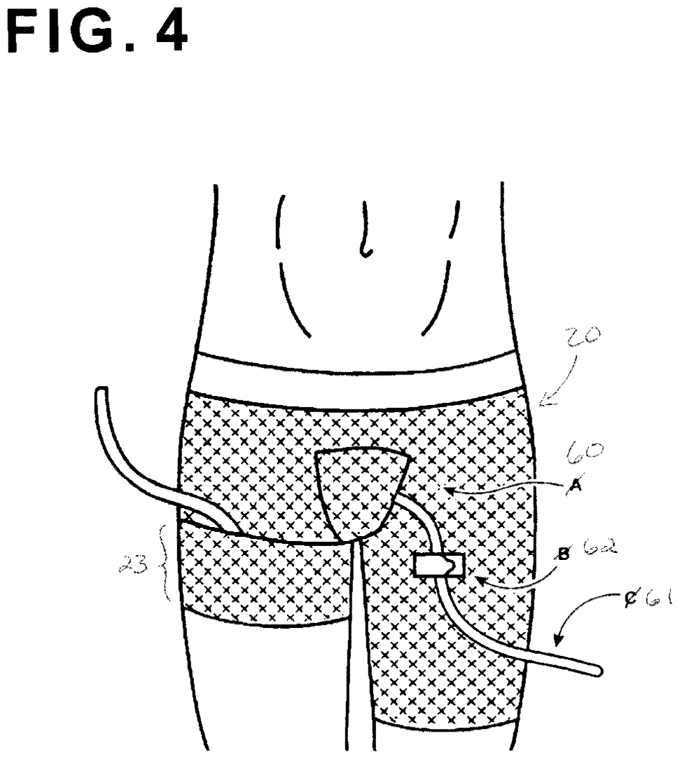

As noted above, the garment and STP could be made from a variety of materials, however in one embodiment it is contemplated that the entire STP garment could be made of elastic stockinette material giving the entire garment a form fitting nature when worn. As shown and described below in conjunction with FIGS. 3-4B, such an embodiment may be particularly advantageous in the lower extremity giving more form and substance to an obese abdomen that could impede tourniquet placement. It is also contemplated that the stockinette could only be a portion of the STP, the portion where the tourniquet is applied (as shown in FIGS. 1A-1C). However, the remainder of the garment could be a more loose fit for patient comfort. This design may be more practical and comfortable for the upper extremity. Another feature that may be beneficial especially on the upper extremity is to leave the contralateral limb 15 uncovered for blood pressure cuffs or intravenous lines. Finally, the STP can be made reversible so that it can be used for either a left or right sided extremity. Again, on the upper extremity version, it may be advantageous to place one or more openings 14 within the STP garment. When such one or more openings are over a patient's torso, such a feature may be useful to allow placement of cardiac monitoring leads or other medical devices and/or equipment.

It should be appreciated that in embodiments, STP 12 may be separate from remaining portions of garment 20 (i.e. the STP may be provided as a stand-alone device or arm sleeve).

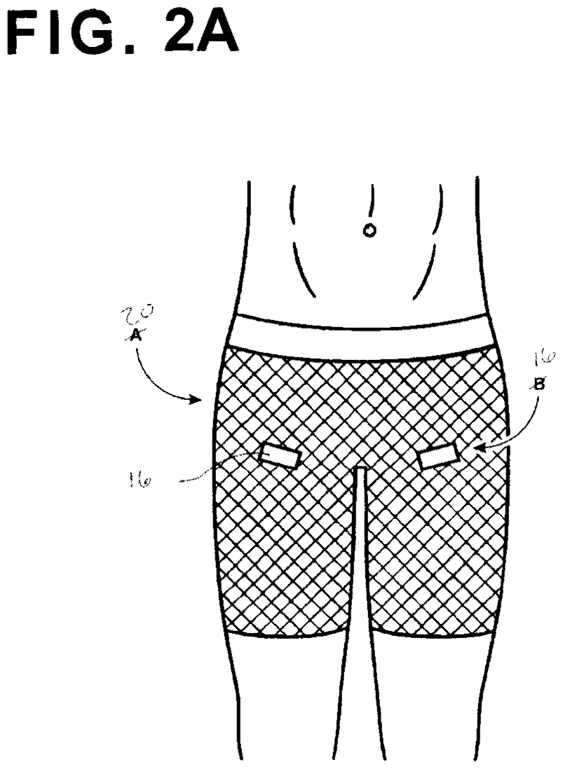

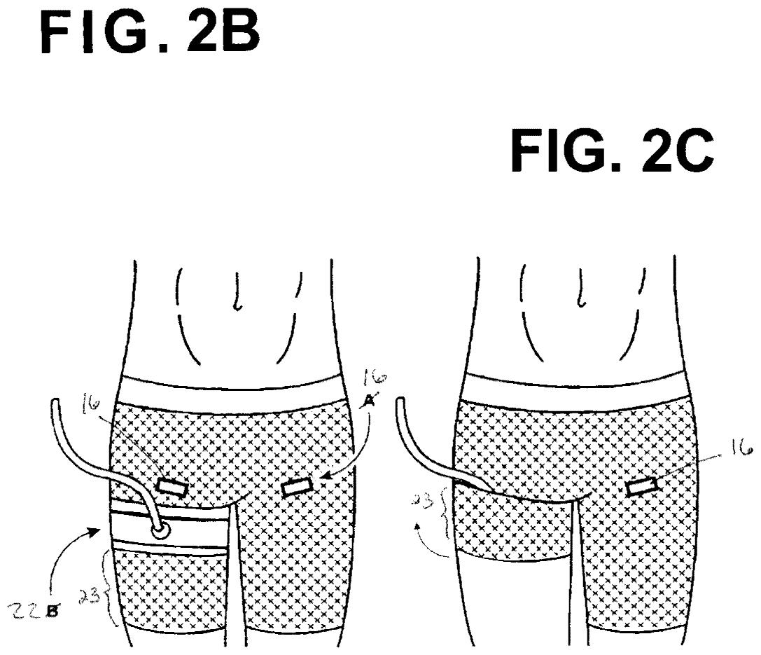

Referring now to FIGS. 2A-2C, in which like elements are provided having like reference designations throughout the several views, a garment 20, here a shorts-type garment, comprises a secure tourniquet protector worn by a patient on a lower extremity (in this case, the secure tourniquet protector may be disposed over leg and/or hip regions of the patient).

In this example embodiment, the entire garment 20 comprises or is provided from a material suitable to act as a secure tourniquet protector. As shown in FIGS. 2A-2C, the secure tourniquet protector is disposed on the lower extremity over the thigh approximately down to the knee.

In this example embodiment, the secure tourniquet protector extends from a patient's waste region to approximately the knee. In embodiments, the secure tourniquet protector extends from a portion of the patient at, above or below the patient's waist region to a portion of the patient at, above or below the knee. The particular length of secure tourniquet protector 20 is selected depending upon the needs of the particular application. In general, the secure tourniquet protector 20 is provided having a length, shape and dimensions selected to suit the needs of a particular patient and a particular application. For example, in embodiments the garment 20 may be provided in a range of different sizes (including, but not limited to, extra-small, small, medium, large, extra-large). After reading the disclosure provided herein, those of ordinary skill in the art will appreciate how to select garment and STP sizes (e.g., an STP length, diameter, and other dimensions) to suit the needs of a particular patient and a particular application.

The ability to fold back the form fitting elastic material portion of the garment (e.g., a stockinette portion of the garment) over the tourniquet, secures the tourniquet from moving on the extremity (e.g., the limb). For example, the ability to fold back the form fitting elastic material portion of the garment over the tourniquet, impedes the ability of the tourniquet (and ideally, totally prevents the tourniquet) from sliding down the limb. This may be particularly advantageous in the case of an obese patient or a patient having a cone shaped extremity. Because at least the STP portion of the garment is made of a form fitting, elastic material it conforms to the tourniquet and upper end of the extremity and cannot slip distally.

As illustrated in FIGS. 2B, 2C, once a tourniquet 22 is placed or otherwise disposed on a patient (e.g., on one or more body parts of a patent), an end of the STP (e.g., here a distal end 23 of STP 20) may be everted and folded back on itself proximally covering substantially the entire tourniquet (and ideally covering the entire tourniquet). Thus, the garment (e.g. an STP portion of the garment) extends down an extremity or a patient by a distance which allows an STP (e.g. an STP portion of a garment) to fold back over a tourniquet.

For additional security, once the garment is folded back on itself over the tourniquet (e.g. as illustrated in FIGS. 2B, 2C), it can be further secured by a securing means 21. The securing means may be provided as a hook and loop type fastener (e.g. a tab of Velcro or other hook type fastener), an adhesive strip fastener or a mechanical fastener (e.g. a snap fastener such as may be provide from a pair of interlocking discs) or a button (e.g., a small disk or knob sewn or otherwise attached to a garment, either to fasten the STP being pushed through an opening (e.g. a hole or slit) made for the purpose of coupling one or more portions of the STP or other garment portion. The tourniquet is then secure and standard prepping and draping of the extremity continues in to begin the surgical or other medical procedure.

The ability to fold back the form fitting elastic material portion of the garment (e.g., a stockinette portion of the garment) over the tourniquet, secures the tourniquet from moving on the extremity (e.g., the limb). For example, the ability to fold back the form fitting elastic material portion of the garment over the tourniquet, impedes the ability of the tourniquet (and ideally, totally prevents the tourniquet) from sliding down the limb. This may be particularly advantageous in the case of an obese patient or a patient having a cone shaped extremity. Because at least the STP portion of the garment is made of a form fitting, elastic material it conforms to the tourniquet and upper end of the extremity and cannot slip distally.

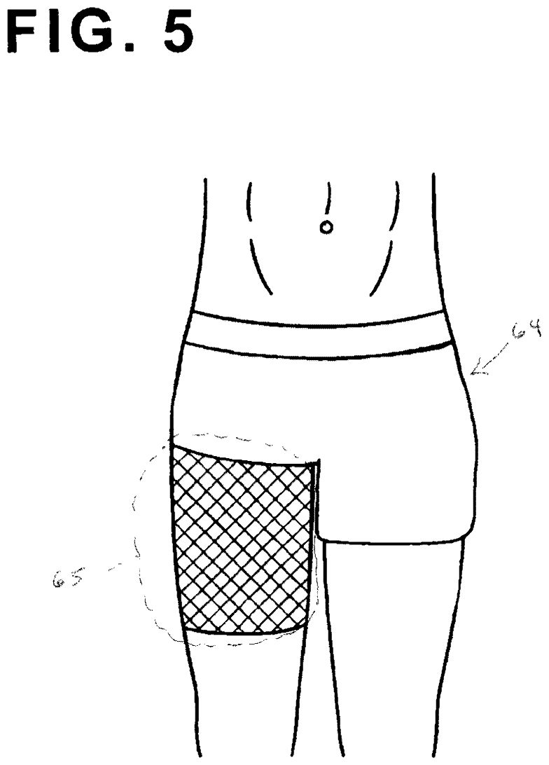

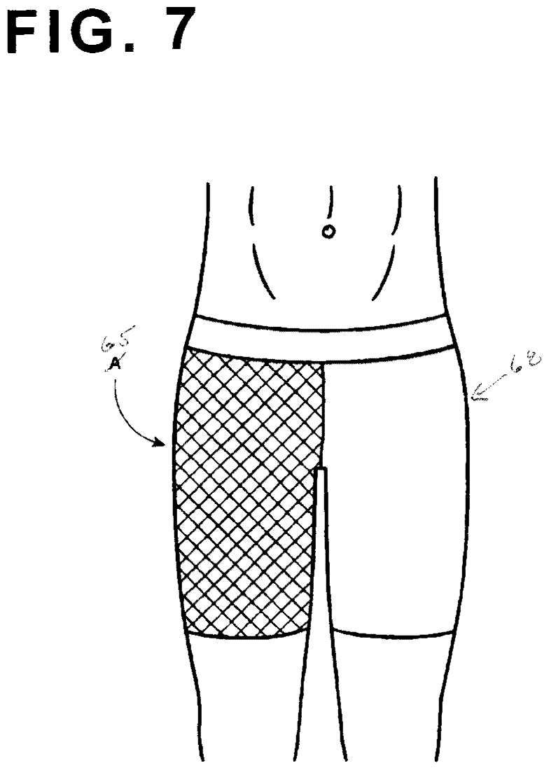

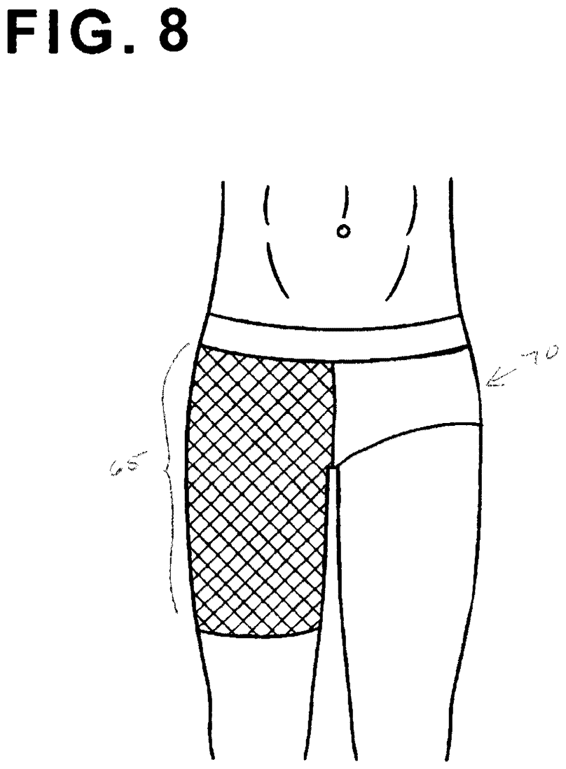

As noted above, the garment and STP could be made from a variety of materials, however in one embodiment it is contemplated that the entire STP garment could be made of elastic stockinette material giving the entire garment a form fitting nature when worn. As shown and described in conjunction with FIGS. 2A-2C, such an embodiment may be particularly advantageous in the lower extremity giving more form and substance to an obese abdomen that could impede tourniquet placement. It is also contemplated that the stockinette could only be a portion of the STP, the portion where the tourniquet is applied (e.g., as shown in FIGS. 1A-1C above and FIGS. 5-8 below). However, the remainder of the garment could comprise a different material (e.g. a natural fiber such as cotton, wool or the like or a synthetic material) selected for ease of manufacture and/or ease of patient donning the garment and/or patient comfort while wearing the garment.

Such a design may be more practical and comfortable for a lower extremity. Another feature that may be beneficial is to leave the contralateral limb uncovered (see e.g., region 24 in FIG. 4B). Finally, the STP can be made reversible so that it can be used for either a left or right sided extremity. Garment 20 may further comprise one or more openings within the STP garment. When such one or more openings are over a patient's body, such a feature may be useful to allow placement of medical devices and/or equipment such as a catheter.

Referring now to FIG. 3, in embodiments an STP 20 may further comprise a region in which padding 50 is disposed (e.g., soft padding). This such padding may disposed or otherwise arranged in an area of the STP corresponding to the area in which a tourniquet would be applied.

In an upper extremity (e.g., as shown in FIGS. 1-1C) padding may be disposed at a superior aspect of the arm. For example, in embodiments, an STP may include a region near the axilla in which padding may be disposed.

In a lower extremity (e.g., as shown in FIG. 3) an STP may include a region in which padding may be disposed at a superior aspect of the thigh near the inguinal area.

It should be appreciated that in embodiments, padding (e.g., padding 50 in FIG. 5) may be an integral part of the STP such that when the garment is donned by a patient, the padding is in place. It should also be appreciated that in embodiments, padding (e.g. padding 50 in FIG. 5) may be a piece which is separate from the STP and arranged over the STP prior to placement of a tourniquet.

Referring now to FIG. 4, in another embodiment, it is possible a planned procedure is one where an extended operative time is expected. In such cases, a catheter is sometimes placed into the bladder to allow urination without soiling the operative site. Thus, in some embodiments, an STP garment may comprise an opening or a removable portion or area 60 that can be opened over or proximate the genital area to allow access for placement of a catheter 61, for example. Further, the catheter itself is often secured to an extremity with an adhesive fastener that is applied to the skin and the catheter is clipped into the fastener as illustrated as region 62. Thus, garment 20 may optionally have a fastening mechanism 62 (or more simply a fastener 62) coupled thereto which may be used to secure a position of a catheter. It should be appreciated that fastener 62 may be provided as an integral portion of garment 20 or may be provided as a part or device separate from the garment and coupled to the garment which may be used to secure a position of a catheter.

In embodiments, fastening mechanism 62 may be provided as a hook and loop fastener configured to secure a device (including, but not limited to a catheter, cardiac leads or any other device or structure as needed) to the garment.

In embodiments, fastening mechanism 62 may be provided as an adhesive strip configured to secure a device (including, but not limited to a catheter, cardiac leads or any other device or structure as needed) to the garment.

In embodiments, fastening mechanism 62 may be provided as a mechanical fastener configured to secure a device (including, but not limited to a catheter, cardiac leads or any other device or structure as needed) to the garment. Such mechanical fasteners may, for example, be provided as a snap fastener (such as may be provide from a pair of interlocking discs) or a button (e.g., a small disk or knob sewn or otherwise attached to a garment and pushed through an opening e.g. a hole or slit in the garment).

Thus, as described it is contemplated that an STP may comprise one or more fastening mechanisms to secure an edge or portion of an STP after it is folded back and/or secure devices used during surgery (e.g. catheters, cardiac leads or any other devices or structures needed during a medical procedure).

As illustrated in FIGS. 2A-4, it is contemplated the entire garment (including STP portion) could be made of a material having a stretchable or elastic characteristic (e.g., stockinette or a stockinette-like material) giving the entire garment a form fitting nature when worn (e.g., as shown and described in conjunction with FIGS. 2A-2C). As noted, such embodiments may be particularly advantageous in the lower extremity giving more form and substance to a large abdominal region of a patient (e.g. an obese abdomen) that could impede tourniquet placement.

It should, however, also be appreciated that as illustrated in FIGS. 5-8 below, it is also contemplated that only a portion of an entire garment could be made of an STP material (e.g., a material having a stretchable or elastic characteristic such as a stockinette or a stockinette-like material). As noted, such embodiments may be useful in some surgical applications in which it is not practical or not possible for a patient to wear a garment.

Referring now to FIGS. 5-8, it is also appreciated the garment and STP could be made from different materials. For example, and as described above, it is contemplated that only a portion of a garment where a tourniquet is to be applied be made from an STP material (e.g. an elastic stockinette material or other appropriate STP material). As illustrated in FIGS. 5-8, this approach gives a portion of an STP garment a form fitting nature. In embodiments when only a portion of the STP garment where a tourniquet is to be applied is provided as stockinette-like material, the remainder of the garment could comprise a different material selected for ease of manufacture and/or ease of patient donning the garment and/or patient comfort.

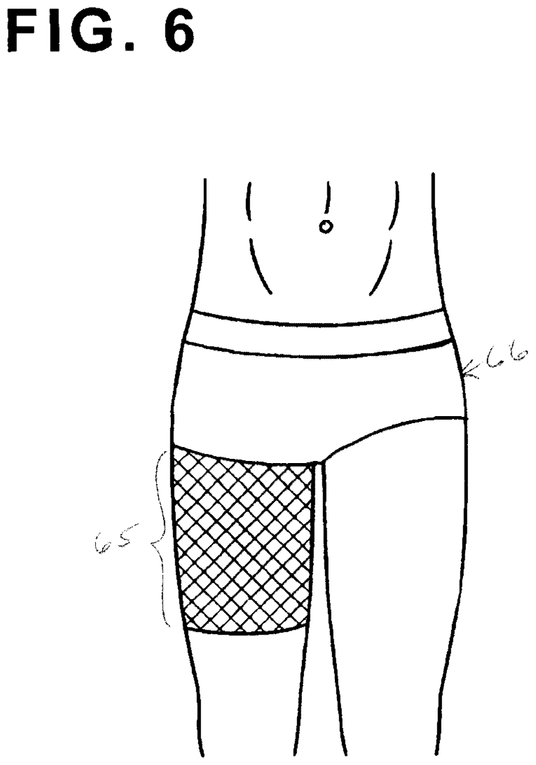

Such a design may be more practical and comfortable for a lower extremity. Another feature that may be beneficial is to leave all or a portion the contralateral limb uncovered (e.g., see FIGS. 5 and 6). Finally, the STP can be made reversible so that it can be used for either a left or right sided extremity.

In FIG. 5 an STP garment is provided as a pair of shorts 64 having a portion comprising an STP 65. In this example embodiment, the secure tourniquet protector 65 extends from a patient's waste region to approximately the knee. In embodiments, the secure tourniquet protector extends from a portion of the patient at, above or below the patient's waist region to a portion of the patient at, above or below the knee. The particular length of secure tourniquet protector 65 is selected depending upon the needs of the particular application. In general, the secure tourniquet protector 65 is provided having a length, shape and dimensions selected to suit the needs of a particular patient and a particular application. For example, in embodiments the garment 64 may be provided in a range of different sizes (including, but not limited to, extra-small, small, medium, large, extra-large). After reading the disclosure provided herein, those of ordinary skill in the art will appreciate how to select garment and STP sizes (e.g., an STP length, diameter, and other dimensions) to suit the needs of a particular patient and a particular application.

FIGS. 6-8 illustrate different variations of STP shorts.

It should also be appreciated that in embodiments, the STP (e.g. STP 65 in FIG. 5) may be separate from remaining portions of garment 64 (i.e., the STP may be provided as a stand-alone device or leg sleeve).

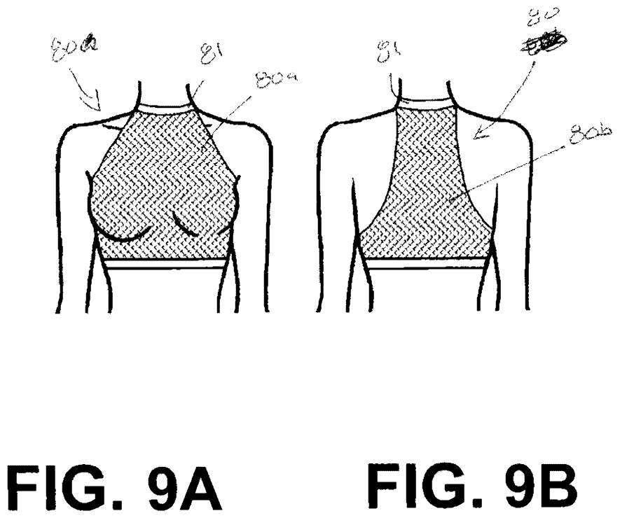

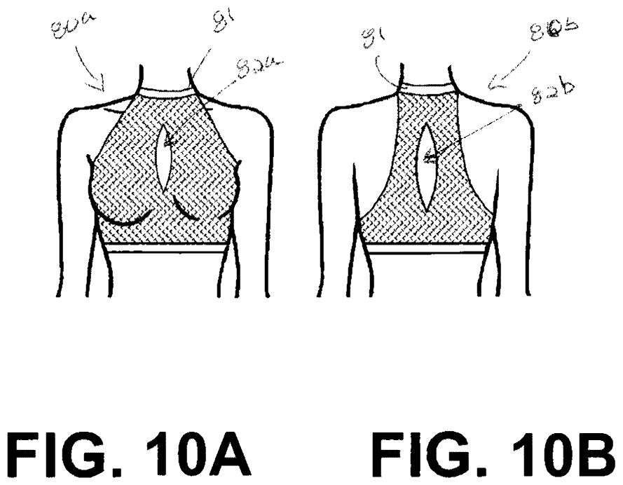

Referring now to FIGS. 9A-10B in which like elements are provided having like reference designations throughout the several views, shown is a peri-operative garment 80 provided as a brassiere having front and back portions 80a, 80b which may be provided from an STP-type material (e.g., a stretchable, elastic material such as stockinette or similar material). In this example embodiment, the front and back garment portions 80a, 80b stay as close to the midline as possible until the material curves laterally to cover the breast area in the front. Garment 80 may then narrow laterally to create a band going around the chest beneath the patient's axilla. These contours are configured to allow for surgical draping of the shoulder and upper extremity,

In this example embodiment, peri-operative garment 80 comprises an optional collar 81 around the neck area to which front and back portions 80a, 80b are attached. In embodiments, collar 81 may be provided from a material which is soft and comfortable.

Common brassieres worn for daily use frequently have metallic parts such as underwires or clasps made of metal. Such metallic parts are not compatible with an operating room environment where devices such as electrocautery are used and could result in skin burns for a patient wearing such a brassiere.

Peri-operative brassiere 80 does not have any metallic parts or features. Rather a peri-operative specific brassiere is provided from all cloth or other material without metallic fasteners, devices or structures of any kind. One goal of the peri-operative brassiere and all of the garments described herein, is to provide coverage of sensitive body parts (e.g. breasts in this example embodiment) for the emotional and physical comfort of the patient, while allowing easy access to the body area needed to perform the medical procedure. Therefore, a form fit design is preferred and is practical. It would also have to not be obstructive to intra-operative monitoring devices like electrocardiogram leads and of course not obstruct proper sterile preparation of the procedure site nor obstruct the procedure itself. Thus, the peri-operative brassiere provides some (and ideally minimal) but adequate and comfortable coverage of the breast area.

As illustrated in FIGS. 10A, 10B, front and/or back sides 80a, 80b of garment 80 may be provided having one or more openings therein 82a, 82b. In such embodiments, the one or more openings may be provided in or about the midline, or elsewhere in the garment. Such openings may optionally be included in the garment 80 to allow access to the skin on the chest for various reasons including, but not limited to placement and/or monitoring of intra-operative electronic monitoring leads.

As described herein, a secure tourniquet protector is a device or garment or a combination of a device and garment that is worn by a patient. Ideally, the STP garment is for a patient to put on. It is designed to accommodate the unique needs of the operating room in regard to placement and securing a tourniquet around an extremity during surgery. The STP facilitates placement and application of the tourniquet saving operating room time and freeing OR personnel for other tasks. At the same time, it also provides security to the tourniquet so that it cannot shift, slide or move from the desired position on the limb. This is helpful in all cases but particularly in cases of an obese patient or with a cone shape to the extremity.

In embodiments, the STP may be separate from a garment (i.e. the STP may be provided as a stand-alone device.

Various embodiments of the concepts, structures and techniques sought to be protected are described. It should, however, be appreciated that alternative embodiments can be devised without departing from the scope of the concepts, structures and techniques described herein.

It is noted that various connections and positional relationships (e.g., over, below, adjacent, etc.) are set forth between elements in the following description and in the drawings. These connections and/or positional relationships, unless specified otherwise, can be direct or indirect, and the described concepts, structures and techniques are not intended to be limiting in this respect. Accordingly, a coupling of elements can refer to either a direct or an indirect coupling, and a positional relationship between elements can be a direct or indirect positional relationship.

As an example of an indirect positional relationship, references in the present description to forming or providing element or structure “A” over element or structure “B” include situations in which one or more intermediate elements or structures (e.g., element or structure “C”) is between element or structure “A” and element or structure “B” as long as the relevant characteristics and functionalities of element or structure “A” and element or structure “B” are not substantially changed by the intermediate element(s) or structure(s).

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a method, article, or apparatus that comprises a list of elements is not necessarily limited to only those elements but can include other elements not expressly listed or inherent to such a method, article, or apparatus.

Additionally, the term “exemplary” is used herein to mean “serving as an example, instance, or illustration.” Any embodiment or design described herein as “exemplary” is not necessarily to be construed as preferred or advantageous over other embodiments or designs.

The term “one or more” and “at least one” are understood to include any integer number greater than or equal to one, i.e. one, two, three, four, etc. The terms “a plurality” and “two or more” are understood to include any integer number greater than or equal to two, i.e. two, three, four, five, etc.

The terms “connection” and “coupled” (including variants thereof) can include an indirect connection or indirect coupling as well as a direct connection or direct coupling. An indirect “connection” or indirect “coupling” means two elements or structures may be connected through one or more other elements or structures. A direct connection or direct coupling means two elements or structures are connected without any other elements or structures therebetween.

The term “direct contact” means that a first element, such as a first structure, and a second element, such as a second structure, are connected without intermediary structures at the interface of the two elements.

References in the specification to “one embodiment, “an embodiment,” “an example embodiment,” etc., indicate the embodiment described can include a particular feature, structure, or characteristic, and also that every embodiment can include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether explicitly described or not.

For purposes of the description here, the terms “upper,” “lower,” “right,” “left,” “vertical,” “horizontal, “top,” “bottom,” and derivatives thereof relate to the described elements, structures and methods, as oriented in the drawing figures. The terms “overlying,” “atop,” “on top, “positioned on” or “positioned atop” mean that a first element, such as a first structure, is present on a second element, such as a second structure, where intervening elements such as an interface structure can be present between the first element and the second element.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately,” “about,” “substantially” and “substantially equal” may be used to mean within ±20% of a target value in some embodiments, within ±10% of a target value in some embodiments, within ±5% of a target value in some embodiments, and yet within ±2% of a target value in some embodiments. The terms “approximately,” “about,” “substantially” and “substantially equal” may include the target value. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

It is to be understood the disclosed subject matter is not limited in its application to the details of construction and to the arrangements of the components set forth in the above description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate the conception, upon which this disclosure is based, may readily be utilized as a basis for the designing of other structures, methods, and systems for carrying out one or more of the purposes of the disclosed subject matter. Therefore, the claims should be regarded as including such equivalent constructions insofar as they do not depart from the spirit and scope of the disclosed subject matter and/or thew concepts described herein.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter and the concepts described herein.

What is claimed is:

1. A secure tourniquet protector configured to be worn by a patient to aid in placement and securing a tourniquet around an extremity of a patient during surgery.

2. A peri-operative undergarment comprising a secure tourniquet protector configured to be worn by a patient to aid in placement and securing a tourniquet around an extremity of a patient during surgery having a size shape and material compatible with a medical procedure to be performed.

3. A peri-operative undergarment having a size shape and material compatible with a medical procedure to be performed and configured to aid in placement and securing a tourniquet around an extremity of a patient during surgery having a size shape and material compatible with a medical procedure to be performed.
